(19) United States
(12) Patent Application Publication
(10) Pub. No.: US 2009/0324723 A1
(43) Pub. Date: Dec. 31, 2009

(54) Method of prophylaxis of infection

(76) Inventors: Grant Thomas Rawlin, Kilmore East (AU); Gottfried Lichti, Essendon (AU); Roy Michael Robins-Browne, Templestowe (AU); Brian David Muller, Bentleigh (AU)

Correspondence Address: Gerstenzang, William C ., 875 THIRD AVE,, 8TH FLOOR, NEW YORK, NY, 10022, US

(21) Appl. No.: 10/992,478

(22) Filed: Nov. 19, 2004

(30) Foreign Application Priority Data
May 21, 2002 (AU) PS2455

(51) Int. Cl. A61K 9/14; A61K 39/40; A61K 9/12; A61P 11/00; A61P 37/04

(52) U.S. Cl. 424/489; 424/164.1; 424/45

(57) ABSTRACT

A method for prophylaxis of infection of the respiratory tract of a subject by pathogenic airborne bacteria the method comprising administering to the subject by inhalation binding proteins directed against the bacteria. the pathogenic bacteria is a bacteria which survives inside phagocytes and the binding proteins are directed against said bacteria which survives inside phagocytes. The binding proteins comprise antibodies or antibody fragments directed against said bacteria which survives inside phagocytes. The binding proteins are selected from the group consisting of polyclonal antibodies, monoclonal antibodies, F(ab) fragments, F(ab′)₂ fragments, antibody tip fragments, chimeric and humanized antibodies and fragments and recombinant antibodies and fragments.

This patent relates to the prevention in humans and other mammals of symptoms arising from the presence of air-borne pathogenic bacteria. Such bacteria include Yersinia spp, Mycobacterium spp, Brucella spp, Bacillus anthracis, Chlamydia pneumoniae, Coxiella burnetii and Legionella pneumophila.

### BACKGROUND

Kollberg (WO9841235) teaches the use of avian polyclonal antibodies against the bacteria Pseudomonas aeruginosa to treat respiratory tract infections caused by this bacteria. The antibody was applied topically to the respiratory tract of children suffering cystic fibrosis. The bacteria was present prior to treatment commencing. The beneficial effect of the antibody was measured by isolating bacteria from the sputum. Prolonged treatment was associated with reduced bacterial counts. Pseudomonas aeruginosa does not survive within phagocytic cells. Kollberg did not demonstrate sustained prophylaxis since the antibodies were always applied after the bacteria were present.

Ramisse et al (Journal of Infectious Disease, May 1996, 173(5) 1123-8) teaches the use of polyclonal antibodies against Streptococcus pneumoniae to treat respiratory infections in mice caused by this bacteria. The antibody and its fragment were applied either intravenously or intranasally after the mice were infected with the bacteria. Streptococcus pneumoniae does not survive within phagocytic cells. Sustained prophylaxis associated with the use of antibodies was not suggested.

Cheng et al (Infectious Immunity April 2001; 69(4): 2302-8) teaches the use of polyclonal antibodies against group B Streptococcus bacteria to increase opsonisation and phagocytosis of this bacteria. Opsonisation is the process where an antibody bound to a bacteria stimulates the activity of phagocytic cells in the locality. This was measured by increased killing of the bacteria by macrophages in cell culture. The increase of phagocytosis is presumably beneficial in vivo however no in vivo experiments were reported. Streptococcus bacteria do not survive in phagocytic cells. Sustained prophylaxis was not demonstrated.

De Hennezel et al (2000, Antimicrobial Agents and Chemotherapy, 45(1):316 teaches the use of polyclonal antibodies against Streptococcus pneumoniae to treat infections in mice caused by this bacteria. The antibody was delivered intranasally and parenterally. The beneficial effect was measured by recording lethality and counting bacteria in lung homogenates. Streptococcus pneumoniae does not survive in phagocytic cells. De Hennezel et al did not demonstrate sustained prophylaxis since the antibodies were applied after the bacteria were present.

Collins (U.S. Pat. No. 4,994,269) teaches the use of a monoclonal antibody against Pseudomonas aeruginosa to prevent and treat respiratory disease caused by that bacteria. The intranasal use of polyclonal and monoclonal antibodies is described and the beneficial effect was recorded by measuring lethality and counting bacteria in lung homogenates. Pseudomonas aeruginosa does not survive in phagocytic cells. Collins gave the antibody preparations 20 minutes before challenge with the bacteria—this is does not suggest a sustained prophylactic function because it would require dosing to happen very frequently to provide a clinically useful effect.

Eyles et al (Vaccine April 1998; 16(7):698-707) teaches the use of an active intranasal vaccine made of bacterial proteins to stimulate the body's own immune system and to prevent disease caused by the respiratory form of Yersinia pestis. Yersinia pestis does survive within phagocytic cells. Particular success was seen when cholera toxin B subunit was used as a strong mucosal adjuvant to stimulate an immune response in the mice. The preparation in the above treatment (vaccination) does not comprise antibodies.

Di Genaro et al (1998, Microbiological Immunology; 42(11):781) teaches the use of an active intranasal vaccine made of bacterial proteins to stimulate the body's own defense system and to prevent disease caused by a respiratory form of Yersinia enterocolitica in mice. Yersinia does survive within phagocytic cells. The preparation in the above-treatment (vaccination) does not comprise antibodies.

For bacteria that survive in phagocytic cells (eg Yersinia) the literature does not teach that the topical application of antibodies would result in a useful clinical result. The following concepts also teach away from the topical application of antibodies in managing respiratory disease caused by bacteria that survive in phagocytic cells:

Antibodies which bind with bacteria increase the phagocytosis of those bacteria by immune cells such as macrophages. This process (also known as Fc receptor-mediated phagocytosis) is well described in many medical texts and is known as ‘opsonisation’ (Huber et al, Journal of Immunology, Jun. 15, 2001; 166(12): 7381-8). A consequence of opsonisation is as follows: for bacteria which survive within phagocytes, antibody treatments will be counter-productive because the antibodies will encourage the bacteria to enter a safe haven (the phagocytes).

The known vaccines for protection of mammals against air-borne bacteria that cause respiratory must be given at least weeks before possible exposure to the pathogen of concern to allow the immune system of the body to respond to the vaccine.

### SUMMARY

We have made the surprising discovery that sustained prophylaxis against bacterial infection in the mammalian respiratory tract can be achieved by inhalation of binding proteins. By sustained prophylaxis we mean a clinically useful period of prophylaxis of at least one hour and preferably at least 3 hours.

Accordingly we provide a method of prophylaxis of bacterial infection of the respiratory tract of a patient, the method comprising inhalation by the patient of, binding proteins.

The invention further provides the use of binding proteins in preparation of a medicament for prophylaxis of bacterial infection of the respiratory tract by administration of the medicament to the respiratory tract.

In a further aspect the invention provides a composition for prophylaxis of bacterial infection of the respiratory tract, the composition comprising binding proteins and a propellant for delivering the composition as an aerosol for inhalation.

In yet a further embodiment the invention provides an inhaler product for prophylaxis of bacterial infection of the respiratory tract comprising a pressurized container enclosing a mixture of a binding protein composition and a propellant, preferably an HFC propellant.

### DETAILED DESCRIPTION

In one preferment the bacteria are bacteria which survive inside phagocytes and the binding proteins are directed against such bacteria, for example Yersinia spp, Mycobacterium spp, Brucella spp, Bacillus anthracis, Legionella pneumophila, Coxiella burnetii and Chlamydia pneumoniae.

It is preferred that the binding proteins comprise antibodies or antibody fragments directed against the bacteria. Examples of antibodies and antibody fragments include polyclonal antibodies, monoclonal antibodies, F(ab) fragments, F(ab)2 fragments, antibody tip fragments, chimeric and humanized antibodies and fragments, and recombinant antibodies and fragments.

It is preferred that the antibody preparations are affinity purified. Preferably the binding proteins are formulated in conjunction with other agents which protect the function of the antibodies in hostile environments. Examples of such agents are mammalian colostrum and colostrum extracts which have been described in International Patent Application No. PCT/AU03/00348, the contents of which are incorporated by reference.

In one preferment the antibodies are taken from bovine colostrum or the yolk of a bird egg. Example, antibodies from hyperimmune colostrum (or hyperimmune egg yolk) may be used. It will be understood by those skilled in the art having regard to the above disclosure that suitable binding protein may be prepared as hyperimmune colostrum (or egg yolk) by immunizing a mammal (or bird) with antigen derived from the pathogen.

The method described in this patent gives immediate sustained protection (for at least 1 hour) making it suitable for use immediately before entering a high-risk area.

In one embodiment the invention provides an inhaler composition comprising a mixture of binding protein or proteins and a propellant. The composition may further-comprise excipients in addition to the carrier.

The propellant may be a fluorocarbon propellant such as a CFC, HCFC or HFC. Hydrofluorocarbon (HFC) propellants are particularly preferred. Examples of suitable hydrofluorocarbon propellants include HFC-134a and HFC-227.

The carrier for the binding protein is preferably essentially free of water. The carrier may be a finely divided particulate material or a liquid. In many cases the binding protein is moisture sensitive. The composition of the binding protein may be a solution or dispersion in the propellant and the composition may include further solvents such as lower alkanols (eg ethanol) glycerol, lower alkylene glycols or mixture.

The inhaler device of the invention preferably includes a container which maintains the formulation under pressure and is impermeable to the ingress of moisture. The inhaler device may be a single use inhaler or multidose inhaler. A multidose inhaler may be provided with a drug metering valve. The inhaler may contain a moisture absorbing material.

In an alternative embodiment the composition may comprise a finely divided solid composition comprising the binding protein and a liquefied gas. The liquefied gas is preferably an inert gas such as nitrogen or a noble element. An example of a suitable method of formulation in liquid gas is described by Meekka et al in U.S. Pat. No. 6,378,518.

In yet another embodiment the binding protein may be in finely divided form comprising particles of for example from 20 nm to 100 microns and preferably 50 nm to 10 microns. The finely divided composition may include anticaking agent.

The inhaler device of the invention may include a compartment containing a powder, a passage for providing an air-stream by inhalation of the user and means for releasing the binding protein powder formulation into the air stream.

The inhaler device may comprise a multiplicity of chambers. In one embodiment for example the device includes one chamber containing a binding protein in dry finely divided form and a second chamber containing a propellant or carrier. The device comprises means for providing mixing of the contents of the first and second chambers. For example, the chambers may be separated by a frangible wall which in operation is breached to provide mixing of the finely divided dry binding protein and the carrier or propellant. Such an arrangement may be particularly useful where the binding protein has a limited shelf life in the carrier or propellant. In this embodiment the inhaler may include means for rupturing the frangible wall. The frangible wall when used may be ruptured by a variety of means such as a piercing plunger or by relative rotation of the different section of an inhaler each provided with one of said chambers.

In a further embodiment of the inhaler of the invention the composition of the binding protein includes a liquid carrier which may be premixed with the binding protein or mixed therewith prior to inhalation and the inhaler,comprises a spray nozzle and mechanical means for providing delivery of liquid binding protein mixture to the spray nozzle to form an aerosol.

The dose of binding protein required to provide prophylaxis of infection will depend on the particular bacteria and the risk and of exposure. Typically the dose will be in the range of from 0.1 to 100 milligrams per kilogram of bodyweight of the individual in whom infection is to be prevented. More preferably the dose will be in the range of from 0.8 to 80 mg/kg.

The subject to be treated may be a human or lower animal subject.

The invention will now be described with reference to the following examples. It is to be understood that the examples are provided by way of illustration of the invention and that they are in no way limiting to the scope of the invention.

### EXAMPLE 1

The results of Example 1 are discussed with reference to the attached drawings. In the drawings:

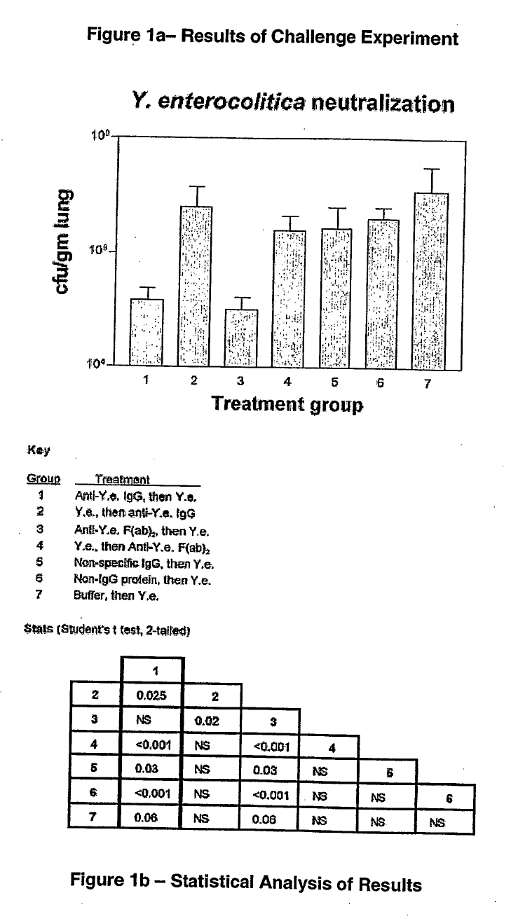

FIG. 1(a) is a chart comprising the effect of prophylactic treatment in accordance with the invention with controls; and

FIG. 1(b) examines the statistical significance of the results illustrated in FIG. 1(a).

Pernasal administration of specific antibody provides sustained immuno-protection of mice against respiratory challenge with a intracellular bacterial pathogen (Yersinia enterocolitica) when given before the bacterial challenge.

In this example Yersinia enterocolitica serves as a model for Yersinia pestis.

### INTRODUCTION

The aim of this study was to determine if pernasally administered polyclonal antibodies obtained could provide sustained protection when challenged with an inhaled bacterial pathogen.

The mouse model used in this study involved pernasal inoculation with Yersinia enterocolitica, a pathogen that is able to colonies and cause serious pneumonia in the lungs of mice. Yersinia bacteria survive within phagocytic cells.

For this study, purified rabbit antibodies (whole IgG or F(ab′)₂ antibody fragments) directed against Y. enterocolitica and a variety of control materials were given pernasally to anesthetized mice, either 3 hours before or 3 hours after the intranasal challenge with a suspension of 5×10⁶ colony-forming units (CFU) of living Y. enterocolitica bacteria. The extent of the protection against infection given by the antibodies and their fragments was determined from measurement of bacterial clearance 24 hours after infection.

### EXPERIMENTAL PROCEDURES

Bacteria

A virulence plasmid-bearing strain of Y. enterocolitica O:8 (strain 8081) was cultured in Tryptone Soy broth (Oxoid) for 24 hr at 28° C. Cells were centrifuged and then washed several times following suspension in sterile PBS. Final suspension was prepared to give 5×10⁶ CFU in 50 μl of sterile PBS. The actual number of bacteria in the inoculum was determined by plating a sample onto Brain Heart Infusion plates (Oxoid) and counting the CFU after incubation for 24 hr at 28° C.

Mice

Groups of five male Black Ten mice (6-8 week old) were kept in separate cages and were infected within a biosafety cabinet to minimize the airborne spread of Y. enterocolitica. Prior to the administration of antibodies and bacteria, mice were anesthetized briefly with inhaled Penthrane (methoxyflurane). Antibodies and bacteria were administered by placing 50 μl of the appropriate control solution, antibody preparation or bacterial suspension onto the nares of the anesthetized mouse. The mouse inhaled the drop and the animal was then allowed to recover. Animals were observed during the recovery period until the righting reflex returned.

Treatment Groups

As detailed in Table 1, test mice received 100 μg of either purified whole rabbit immunoglobulin (IgG) to Y. enterocolitica (0:8) or the F(ab′)₂ fragment of the IgG. Antibodies were given pernasally (in 50 μl solution) to anesthetized mice in each test group (10 mice per test group). Antibodies were given either 3 hours before or 3 hours after intranasal challenge with a suspension of 5×10⁶ CFU of Y. enterocolitica serogroup. Control groups of mice (5 mice per control group) received either 50 μl PBS buffer, 100 μg of purified IgG from unimmunized rabbits (Non-specific IgG) or 100 μg of a pool of serum proteins from unimmunized rabbits, after removal of IgG using Protein A chromatography (Non-IgG proteins). An additional group of 5 control mice were given whole IgG, without a bacterial challenge, to assess any histopathological effect of the antibody on the lungs.

TABLE 1
Treatment Groups

| | | | | Timing of |
|---|---|---|---|---|
| | | | | administration of test |
| | No. of | | | material -before or |
| Group No. | mice | Test material | Dose | after infection |
| 1. Test | 10 | Whole IgG | 100 μg | 3 hr before |
| 2. Test | 10 | Whole IgG | 100 μg | 3 hr after |
| 3. Test | 10 | F(ab′)₂ Fragment | 100 μg | 3 hr before |
| 4. Test | 10 | F(ab′)₂ Fragment | 100 μg | 3 hr after |
| 5. Control | 5 | Non specific IgG | 100 μg | 3 hr before |
| 6. Control | 5 | Non-IgG protein | 100 μg | 3 hr before |
| 7. Control | 5 | Buffer | N/A | 3 hr before |
| 8. Control | 5 | Whole IgG | 100 μg | No Infection |

Measurement of Bacterial Clearance from the Lung

A comparison of bacterial clearance was made 24 hr after infection to determine the extent of the bacterial clearance induced by the test and control materials.

After mice were killed using inhaled CO₂, the cranial left lobe of the lung was removed aseptically and placed in a preweighed bottle containing 1 ml of sterile PBS. The lobe was weighed and then homogenized in the PBS. Serial dilutions were plated on MacConkey agar to estimate counts of the recovered bacteria.

Histopathological Studies

The cranial right lobe of the lung was removed from each mouse, together with the heart, kidney, spleen and part of the liver and fixed in 10% formalin. These tissues were dehydrated in alcohol, embedded in paraffin and sections were stained in haematoxylin-eosin. Lung infection was identified by inflammatory cell infiltration into alveoli, bronchiolitis and loss of normal histoarchitecture. In comparison, lung protection was evaluated by the presence of normal histoarchitecture.

Statistics

Data from the estimates of recovered bacteria in the cranial lobe of the lungs were analyzed using Student's 2-tailed t test. The estimates of CFU were transformed logarithmically to normalize their distribution and reduce the variance. A P value of <0.05 is considered statistically significant.

### RESULTS

Bacterial Clearance from the Lung After Passive Immunization

As shown in the attached FIG. 1a, bacterial clearance from the lungs of treated mice (groups 1 and 3) was highly significant when compared to control mice (P<0.001 vs. group 6 and P<0.05 vs. group 5). These results indicate that pernasal administration, 3 hours before infection, of either the whole IgG of rabbit antibodies to Y. enterocolitica or the F(ab′)₂ fragment of the IgG, significantly reduced the numbers of Y. enterocolitica recovered from the lung.

This protection surprisingly occurred only when the antibodies were given 3 hr before the bacterial challenge but not when given after the challenge.

From FIG. 1b which shows the structural significance of the results it is clear that treatments 1 and 3 (in which antibody was administered prior to challenge by the pathogen) are significantly better.

Histopathological Studies

There was no evidence of any histopathological effect of the pernasal administration of antibody (without a bacterial challenge) on the lungs of 5 control mice that were given whole IgG.

### EXAMPLE 2

Manufacture of Hyperimmune Serum Against Yersinia spp. in Rabbits.

A virulence plasmid-bearing strain of Y. enterocolitica O:8 (strain 8081) was cultured in Tryptone Soy broth (Oxoid) for 24 hr at 28° C. Bacteria were harvested to yield 5×10⁶ CFU in 50 μl of sterile PBS. The number of bacteria in the sample was determined by plating a sample onto Brain Heart Infusion plates (Oxoid) and counting the CFU after incubation for 24 hr at 28° C.

The bacteria were spun down, washed in PBS and then heat-killed by boiling. The bacteria were again washed and aliquoted into doses corresponding to the following numbers of whole heat killed bacteria: 1×10⁶, 4×10⁶, 8×10⁶, 2×10⁷, 4×10⁷, 1×10⁸. These doses correspond to each vaccine dose in sequence.

1 ml of vaccine was injected into the thigh muscle of 3 rabbits twice a week for 3 weeks. 6 vaccinations were given to each rabbit.

Blood was harvested from the ear vein of the rabbits. After harvesting, the blood was allowed to clot and the hyperimmune serum was removed, aliquoted and frozen at −20° C.

### EXAMPLE 3

Manufacture of F(Ab2) Fragments Against Yersinia spp.

Immunoglobulin was purified from rabbit antiserum to Y. enterocolitica (O:8), using Protein-A chromatography on a Millipore ProSep-A affinity column, with PBS pH 7.4 as the running buffer. Immunoglobulin was eluted with 0.1M Glycine/HCl pH 3.0 buffer, neutralized by addition of 1M Tris solution and then dialyzed against PBS pH 7.4. Protein-A chromatography was also used to purify non-specific immunoglobulin from unimmunized rabbit serum for use as “Non-specific IgG” control.

The F(ab′)₂ fragment of the purified rabbit immunoglobulin to Y. enterocolitica (O:8) was prepared by digestion with immobilized pepsin (Pierce Chemical Co.) to remove the crystallizable (Fc) region of the antibody molecule. Prior to digestion, purified immunoglobulin was dialyzed against 20 mM sodium acetate buffer at pH 4.5 and adjusted to a concentration of 10 mg/ml. Immunoglobulin was then incubated with immobilized pepsin at 37° C. for 4 hrs. Digestion was stopped by centrifugation of the incubation mixture to remove the immobilized pepsin and adjustment of the pH of the supernatant to 7.2. The F(ab′)₂ fragment was purified from undigested IgG and whole Fc fragments by re-application over a ProSep-A affinity column. Results from the mice receiving the antibody fragments were used to indicate if passive immunity required intact immunoglobulin or only the antigen-binding portion. All antibody preparations and control solutions were adjusted to a protein concentration of 2 mg/ml in PBS before administration to mice.

### EXAMPLE 4

Hyperimmune Colostrum Against Anthrax

Live anthrax vaccine (STERNE strain supplied by Fort Dodge Animal Health a division of Wyeth, located at Overland Park, Kansas City, Kans., U.S.A.) was used to make hyperimmune-bovine colostrum using the following method.

Cows are immunized by a registered veterinarian with a 1 m injection of the STERNE strain vaccine as supplied into the muscle tissue on the side of the neck. Up to 5 injections are given at 2 weekly intervals during months 6 to 8.5 of gestation, ceasing 1 month before parturition. Test bleeds are taken from a selection of the immunized cows and assayed to determine the level of specific antibodies. Results of these assays are used to determine if a satisfactory titre has been achieved.

Hyperimmune colostrum was harvested and processed according to the method of Example 2 of International Application No. PCT/AU03/00348.

The following diagram shows the principles used to take colostrum and convert it to a processed form.

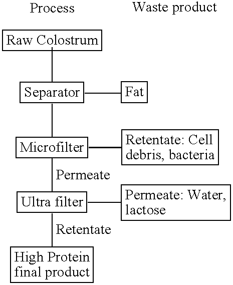

The raw colostrum is collected from dairy cows most preferably at the first milking after calving. The colostrum is stored at 4° C. on farm and then transported either for longer term storage at −20° C. or sent directly to wet manufacturing.

The raw colostrum is warmed to approximately 37° C. and then skimmed with a rotary milk separator to remove fat. The resultant liquid may be pasteurized or microfiltered with a 7-10 micron ceramic filter system (to remove bacteria and debris. The liquid is then Ultrafiltered (for example in a Abcor 10 m² Ultrafiltration plant) to remove a majority of the water, lactose and electrolytes leaving a high protein concentrate. The resultant high protein concentrate is further processed preferably by lyophilization (freeze-drying) or spray-drying.

The above method yield a processed bovine colostrum powder. This product is suitable for inclusion in therapeutic goods.

### EXAMPLE 5

Testing of Hyperimmune Colostrum for Anthrax Binding Capacity.

An Enzyme Linked Immuno-sorbent Assay was created using a recombinant protein form of Protective Antigen of Bacillus anthracis (PA), sourced from SAPHIRE LABS, an agent of AVANT Therapeutics of Massachusetts, U.S.A., part of the Collier Group.

A test liquor was made by mixing the above bovine colostrum extract powder in water to provide a 2% (by weight) aqueous mixture. This was tested alongside PBS (negative) and a standard mouse monoclonal antibody with a significant affinity for Protective Antigen (Bacillus anthracis) using the following assay method. Primary reaction time: 15 minutes.

### ASSAY METHOD

Method for testing colostrum from cows vaccinated with anthrax vaccine for anti-PA antibodies. (PA refers to protective antigen to B. anthracis).
1. One hundred microlitres (100 ul) of a 0.05M Carbonate-bicarbonate buffer containing 1 ug of PA/ml was added to the wells of a 96-well microtitre plate, and the plate was incubated overnight at room temperature.
2. The plate was washed (×3) with PBS/0.05% Tween 20.
3. Three hundred microlitres (300 ul) of 3% skim milk in PBS/0.05% Tween 20 were added to each well containing PA to block any sites that did not bind PA. The plate was incubated at room temperature for 1 hr.
4. The plate was washed as above.
5. One hundred microlitres (100 ul) of a 1 in 500 dilution of a 20 mg/ml solution of a colostrum sample was added to each well containing PA. The plate was incubated at room temperature for 1 hr.
6. The plate was washed as above.
7. One hundred microlitres (100 ul) of a 1 in 2000 dilution of a goat anti-bovine alkaline phosphatase conjugate was added to each well, and the plate was incubated at room temperature for 1 hr.
8. The plate was washed (×3) with PBS/0.05% Tween 20 and once (×1) with distilled water.
9. One hundred microlitres (100 ul) of a p-nitrophenyl phosphate substrate was added to each well, and the plate incubated at room temperature for 1 hr.
10. Finally, the plate was read at 405 nm on an ELISA reader.

The plate also contained a negative (same procedure as the test samples except that 100 ul of 3% skim milk in PBS/0.05% Tween 20 was added instead of the colostrums sample) and positive control (same procedure as test samples except 100 ul of a 1 in 1000 dilution of a mouse anti-PA monoclonal antibody was added as the sample and a goat anti-mouse alkaline phosphatase conjugate was used instead of the anti-bovine conjugate.

All dilutions were made in 3% skim milk in PBS/0.05% Tween 20.

Results

Assay results are shown in Table 2.

TABLE 2

| | Optical |
|---|---|
| Sample | Density |
| Bovine Colostrum Extract (raised against Bacillus anthracis) | 3.7 |
| Anti-PA monoclone (positive control) | 4.2 |
| Phosphate buffered saline (negative control) | <0.1 |

The affinity for the bovine colostrum extract for PA is shown by the high optical density which is (a) similar to the positive control and (b) significantly greater than the negative control.

1. A method for providing passive immunity in the respiratory tract of a subject against pathogenic airborne bacteria selected from the group consisting of Yersinia and Bacillus anthracis, said method comprising administering to the subject by inhalation an effective amount therefor of a polyclonal antibody or a fragment thereof which binds the bacteria.

2-3. (canceled)

4. The method according to claim 1 wherein the antibody or fragment thereof is selected from the group consisting of polyclonal antibodies, F(ab) fragments, F(ab′)₂ fragments, and antibody tip fragments.

5. The method according to claim 1 which further comprises affinity purifying the antibody or fragment thereof prior to administering the antibody or fragment thereof to the subject.

6. The method according to claim 1 wherein the antibody or fragment thereof is administered in conjunction with a protective agent selected from mammalian colostrum and extracts thereof.

7. The method according to claim 1 which further comprises raising the antibody or fragment thereof by vaccination of a mammal and collection of colostrum or by vaccination of an egg yolk or an egg.

8. The method according to claim 1 which further comprises preparing the antibody or fragment thereof as a hyperimmune colostrum or hyperimmune egg yolk.

9-10. (canceled)

11. The method according to claim 1 wherein the antibody or fragment thereof is administered by inhalation as an aerosol.

12. A composition for inhalation as an aerosol for providing passive immunity against a bacteria selected from the group consisting of Yersinia and Bacillus anthracis, said composition comprising an effective amount therefor of a polyclonal antibody or a fragment thereof which binds said bacteria.

13. The composition according to claim 12 further comprising a propellant for delivering the composition as an aerosol for inhalation.

14. The composition according to claim 12 further comprising a protective agent for the antibody or fragment thereof selected from mammalian colostrum and extracts thereof.

15. The composition according to claim 13 wherein the propellant is a hydrofluorocarbon propellant.

16. The composition according to claim 12 wherein the antibody or fragment thereof is from hyperimmune colostrum or hyperimmune egg yolk.

17. The composition according to claim 13 further comprising a non-aqueous carrier selected from lower alkanols, glycerol, lower alkylene glycols and mixtures thereof.

18. The composition according to claim 13 in the form of particles of size in the range of from 20 nm to 10 microns.

19. An inhaler product comprising a chamber containing the composition according to claim 12 and a means for providing an aerosol of the composition.

20. The inhaler product according to claim 19 wherein the composition is in the form of a powder and the inhaler product comprises a passage for providing an airstream by inhalation of a user and means for releasing the powder composition into the airstream to form an aerosol thereof.

21. The inhaler product according to claim 19 comprising a propellant and a valve for delivering an aerosol of the composition.

22. The inhaler product according to claim 21 comprising a multiplicity of chambers including a first chamber comprising said composition and a second chamber containing a propellant and the inhaler further comprises means for mixing contents of the chambers prior to inhalation.

23. The inhaler product according to claim 22 wherein the composition comprises a liquid carrier or is mixed with a liquid carrier prior to inhalation and the inhaler comprises a spray nozzle and mechanical means for providing delivery of the liquid to the nozzle under pressure.

24. The method of claim 1 wherein the antibody or fragment thereof is administered prior to exposure to the bacteria.

25. (canceled)

26. A method for providing passive immunity in the respiratory tract of a subject against bacteria selected from the group consisting of Yersinia pestis and Bacillus anthracis, said method comprising administering to the subject by inhalation an effective amount therefor of a polyclonal antibody or fragment thereof which binds the bacteria.

27. A method for providing passive immunity in the respiratory tract of a subject against Bacillus anthracis, said method comprising administering to the subject by inhalation an effective amount therefor of a polyclonal antibody or fragment thereof which binds Bacillus anthracis.

28. A method for providing passive immunity in the respiratory tract of a subject against Yersinia pestis, said method comprising administering to the subject by inhalation an effective amount therefor of a polyclonal antibody or fragment thereof which binds Yersinia pestis.
